(19) United States
(12) Patent Application Publication
(10) Pub. No.: US 2025/0194962 A1
(43) Pub. Date: Jun. 19, 2025

(54) DEVICES AND METHODS FOR TRANSCUTANEOUS BILIRUBIN MEASUREMENT

(71) Applicant: THE UAB RESEARCH FOUNDATION, BIRMINGHAM, AL (US)

(72) Inventors: Charitharth Vivek Lal, Vestavia, AL (US); Vivek Shukla, Birmingham, AL (US); Mohammad Rafiqul Haider, Vestavia, AL (US)

(21) Appl. No.: 18/867,687

(22) PCT Filed: May 17, 2023
(86) PCT No.: PCT/US2023/022468

§ 371 Date: Nov. 20, 2024

Related U.S. Application Data
(60) Provisional application No. 63/345,310, filed on May 24, 2022.

(51) Int. Cl. A61B 5/145; A61B 5/00; A61B 5/1455

(52) U.S. Cl. CPC A61B 5/14546; A61B 5/002; A61B 5/1455; A61B 5/4244; A61B 5/6804; A61B 5/742; A61B 2562/0238

(57) ABSTRACT

Wearable devices for measuring health indicators such as bilirubin are provided. The device includes a skin-facing side including a circuit board. The circuit board includes a spectrometer integrated circuit, at least one light emitting diode (LED) configured to direct light at the subject's skin where the LED is operably connected to a power source, at least one photodetector configured to receive light reflected from the subject's skin, and a microcontroller configured to perform spectral subtraction of said reflected light.

### CROSS-REFERENCE TO RELATED APPLICATIONS

This application claims the benefit of and priority to U.S. Provisional Application Ser. No. 63/345,310, having the title “DEVICES AND METHODS FOR TRANSCUTANEOUS BILIRUBIN MEASUREMENT”, filed on May 24, 2022, the disclosure of which is incorporated herein by reference in its entirety.

### BACKGROUND

Accuracy of noninvasive measurement of bilirubin in neonates has improved in recent years, but the use of transcutaneous bilirubin measurements is currently restricted to health care facilities. Due to expensive machines, transcutaneous bilirubin monitoring from home is unfeasible.

### SUMMARY

Embodiments of the present disclosure provide apparatus for measuring bilirubin and other health indicators in a subject, methods of measuring bilirubin, and the like.

An embodiment of the present disclosure includes a wearable device for measuring bilirubin levels in a subject. The device can include an externally facing side having a display and a skin-facing side comprising at least one light emitting diode (LED). The LED can be configured to direct light at the subject's skin where the LED is operably connected to a power source. The device can also include at least one photodetector configured to receive light reflected from the subject's skin and a processing chip configured to perform spectral subtraction of the reflected light.

An embodiment of the present disclosure also includes a wearable device for measuring health indicators in a subject. The device can include a skin-facing side comprising a circuit board. The circuit board can include a spectrometer integrated circuit, at least one light emitting diode (LED) configured to direct light at the subject's skin where the LED is operably connected to a power source, at least one photodetector configured to receive light reflected from the subject's skin, and a microcontroller configured to perform spectral subtraction of said reflected light. The health indicators can include one or more of: bilirubin levels, pulse, oxygen saturation, breathing rate, and skin temperature.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a diagram illustrating a wearable band for measuring transcutaneous bilirubin in accordance with embodiments of the present disclosure.

FIG. 2 is a chart comparing three biometric signals clearly differentiated across the optical spectrum (from Jacques, [10]).

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. It is to be understood that, unless otherwise indicated, the present disclosure is not limited to particular materials, reagents, reaction materials, components, manufacturing processes, or the like, as such can vary. It is also possible in the present disclosure that steps can be executed in different sequence where this is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

In accordance with the purpose(s) of the present disclosure, as embodied and broadly described herein, embodiments of the present disclosure, in some aspects, relate to devices and methods for transcutaneous bilirubin measurement.

In general, embodiments of the present disclosure provide for methods of detecting and monitoring bilirubin and devices that include transcutaneous bilirubin measurement.

In some embodiments, the device can be a wearable device (e.g., a wristband/mittens, hat, ankle band/socks, or band suitable for wear around the thigh, arm, waist, etc.). The device can be designed to fit an infant wearer. The device can be enclosed so as to avoid loose parts or other safety hazards to an infant.

Bilirubin is a chromophore molecule formed as a product of hemoglobin metabolism.¹ Neonatal bilirubin metabolism is immature and can easily be overwhelmed, which can lead to hyperbilirubinemia and, if severe, can lead to permanent neurological sequelae.² Visual assessment of neonatal hyperbilirubinemia (also referred to as jaundice) is unreliable^{3,4} hence, serum bilirubin measurements are routinely done for neonatal bilirubin level assessment.

Although bilirubin measurements are often associated with neonates, the devices and methods herein can also be applied to subjects in need regardless of age. For example, adults can have hyperbilirubinemia associated with, such as liver failure and/or hepatitis.

Serum bilirubin assessment is invasive and is restricted to health care facilities as it needs blood sampling. Transcutaneous bilirubin measurement (TcB) is a noninvasive method of bilirubin assessment, the accuracy of which has improved over the last few years.⁵ However, the use of transcutaneous bilirubin measurements is currently restricted to health care facilities due to expensive machines, and its use for home bilirubin monitoring has heretofore been unfeasible.

In addition to bilirubin, in some embodiments the device can also measure one or more of oxygen saturation, breathing rate, and skin temperature.

The present disclosure includes a wearable device comprising at least one LED and at least one photodetector configured to optically measure transcutaneous bilirubin of a wearer (e.g., a neonate). Advantageously, the device is wearable and simple to interpret, allowing monitoring outside of a healthcare environment by caregivers with minimal training, such as parents, or even self-monitoring in the case of adult patients. The devices and methods herein can help in the early identification of at-risk neonates and would help in prompt transfer to an appropriate facility. In turn, the risk of poor neonatal outcomes secondary to neonatal hyperbilirubinemia can be decreased. Advantageously, close bilirubin monitoring may also help in the earlier discharge of neonates and reduce overall healthcare costs by shortening the length of hospitalization.

### Example 1

### Bilirubin Measurement

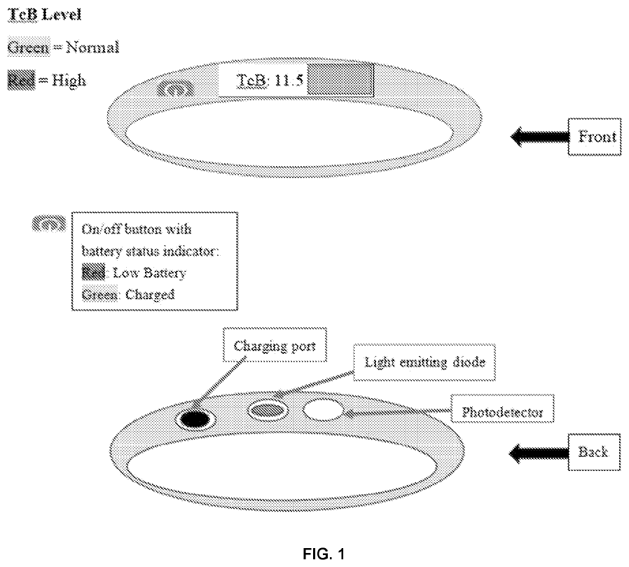

Turning now to FIG. 1, the device can be a wearable device such as a bracelet, anklet, or strap. The wearable device can be adjustable for resizing to fit the wearer or can include a stretchable material (e.g., silicone). A screen can be present on the externally facing side so that a user or caregiver can monitor the readings given by the device. The screen can display information including measurement of transcutaneous bilirubin (TcB) serum concentration μmol/L, a color indicator, a battery indicator, a number of samples taken, or other relevant information as can be envisioned by one of skill in the art. For example, the display could show a TcB value and a red indicator if the level is high. The display can optionally be illuminated for readings in low light or dark environments.

In some embodiments, the device can include an audible alarm or buzzer to indicate early alert in case of emergency. The alarm can be triggered when a value is outside of an expected threshold.

The device can also include a power button or switch.

In various embodiments, the device can include a battery. The battery can be non-rechargeable, such as a watch battery, or a rechargeable battery. A rechargeable battery device can also include a charging port, (e.g. a micro-USB port or other appropriate port) or be configured to charge wirelessly. The charging port can be located on the external side, the skin-facing side, or elsewhere on the device.

The back, or skin-facing side, can include at least one light emitting diode (LED) and at least one photodetector (PD). The LED and PD can be aligned at a certain angle (e.g., similar to a computer wireless-mouse setup) with the skin or the external body part so that the reflected signal can reach the PD after going through the skin environment.

In some embodiments, light from the LED is reflected from the skin, and the corresponding light spectra are captured by the PD.

The device can also include a small processing chip such as a microchip or a microcontroller for processing the signals received by the PD. The microcontroller unit can contain signal processing algorithms for extracting the signal features and low-level assessment of the target analyte (e.g., bilirubin). The computational burden of the microcontroller unit can be optimized considering the limited processing power and memory of the smaller foot-print microchip. The processed signal can then be either saved on micro-SD memory card or wirelessly transmitted to a nearby portable unit.

The portable unit (e.g. a smart phone or custom Raspberry Pi module) can have a relatively powerful processor and higher memory to transfer the computational burden from the wearable device to allow for a wearable monitoring device that is low-power and light-weight. The data from the wearable device transferred through micro-SD memory card or wireless media can be processed using digital signal processing and machine learning algorithms such as power spectral density, correlation, wavelet, neural network, etc. The portable unit can perform the computation to assess the target analyte (e.g., the bilirubin level), transmit back the information to the wearable device, and trigger the alarm signal as necessary. In other embodiments, cloud-based computing can be used.

In various embodiments, the device can contain multi-color LEDs (e.g., red, green, yellow) and transmit a multi-wavelength light. The reflected light can be received by the at least one photodetector or a photodetector array. The number of LEDs and the PDs will depend upon the need, power limit of the wearable device, and the device foot-print. In a specific embodiment, 415 nm and 488 nm LEDs can be used for lower end Green, and 515 nm and 520 nm LEDs for higher end Green light signals. The LED outputs of the wearable device in these wavelengths can be measured and correlated with standard laboratory spectrometer outputs for different bilirubin levels. The correlation process can identify the proper scale factor.

In the portable unit, the determination of transcutaneous bilirubin levels will be accomplished by utilizing the principle of spectral subtraction⁷, wherein the difference in optical densities for light between 450 nm-600 nm regions is measured as a means for determining the yellowness of the wearer's skin while minimizing the influences of skin tone (melanin content) and skin maturity. Spectral subtraction can accurately determine the bilirubin level (peak at 460 nm) with no interference from other chromophores. This method has been used in transcutaneous bilirubin measurement and has been shown to be very accurate.⁸⁻¹¹

The wearable device along with the portable unit will work as a complementary scheme to the laboratory-based medical diagnosis process of bilirubin monitoring. The embodiment can perform an early assessment of bilirubin level, potentially reducing the need for frequent hospital visits and insurance bills. The embodiment can also provide an excellent alternative in remote areas where medical facilities are scarce.

The signal processing system will quantify the serum bilirubin by generating the reflectance spectra expected from the skin with varying absorption and scattering coefficients associated with different biological materials present in different layers of skin using a forward model method. The bilirubin concentration will be estimated using the forward model along with a nonlinear iterative method that adjusts the parameters of the forward model until a best fit is achieved between the simulated and measured spectra. Once the best-fit parameters are obtained, they can be correlated with the experimental conditions associated with the sample preparations¹³. In various embodiments, the signal processing system includes a photodetector connected to a microchip with a processor, where the processor can conduct spectral analysis and providing values. As mentioned above, analyses can alternatively be conducted using cloud computing.

In various embodiments, the device can take spectral readings to determine a bilirubin level at various time intervals adequate for monitoring trends, such as once every 6 hours for 7-day monitoring or hourly for wearers at higher risk for hyperbilirubinemia.

In various embodiments, the spectral readings can be a single reading or multiple readings at each interval. For example, a mean of three readings taken in close succession can be used to indicate the bilirubin level at a given time point.

In some embodiments, the bilirubin readings, trends, and other information can be accessed and tracked in an app to provide monitoring of transcutaneous bilirubin levels. In some embodiments, multiple wearers can be tracked, such as for use by a health care provider. For example, the device can be connected to Wi-Fi and linked to the app, which can be accessed with the username and password that is created when first using the app. The device will relay the data to a central server which can be accessed via an app. There can be a limited number of apps connected to the device. The parents/patient can determine whom to share the link, which can be used to access the patient data. One of the users can be the physician if the parents/patient grant permission. The physician can have a screen that provides a log of multiple patients in a single screen and can communicate with those with abnormal values via the app itself.

In some embodiments, the wearable device as described above can be operated without the need for an external portable computational unit. The wearable device can be Bluetooth low energy (BLE)-enabled and include a BLE-to-WIFI gateway. A circuit board and microcontroller unit can be included such that the circuit board and microcontroller firmware are configured to collect and retransmit the full spectrometer data set, as well as computing metrics all within the device. The microcontroller then uses BLE to transmit the data and metrics to a BLE central device (e.g. the gateway acts as a central hub and transmits BLE signals from the device to the internet). An associated program, such as a smartphone app, can provide user setup options and provide visual indicators for the metrics and data collected from the device to be interpreted by the user (e.g. a parent or caregiver).

### REFERENCES

1. Knudsen A. The influence of the basal yellow colour of the skin at birth on later jaundice meter readings in mature newborn infants. Acta paediatrica (Oslo, Norway: 1992) 1992; 81 (6-7): 494-7.
2. Le Pichon J B, Riordan S M, Watchko J, Shapiro S M. The Neurological Sequelae of Neonatal Hyperbilirubinemia: Definitions, Diagnosis and Treatment of the Kernicterus Spectrum Disorders (KSDs). Current pediatric reviews 2017; 13 (3): 199-209.
3. Aprillia Z, Gayatri D, Waluyanti F T. Sensitivity, Specificity, and Accuracy of Kramer Examination of Neonatal Jaundice: Comparison with Total Bilirubin Serum. Comprehensive child and adolescent nursing 2017; 40 (sup1): 88-94.
4. De Luca D, Zecca E, Zuppa A A, Romagnoli C. The joint use of human and electronic eye: visual assessment of jaundice and transcutaneous bilirubinometry. The Turkish journal of pediatrics 2008; 50 (5): 456-61.
5. Engle W D, Jackson G L, Engle N G. Transcutaneous bilirubinometry. Seminars in perinatology 2014; 38 (7): 438-51.
6. Wang L, Jacques S L, Zheng L. MCML—Monte Carlo modeling of light transport in multi-layered tissues. Computer methods and programs in biomedicine 1995; 47 (2): 131-46.
7. Jacques S L, Saidi I S, Ladner A, Oelberg D. Developing an optical fiber reflectance spectrometer to monitor bilirubinemia in neonates. Laser-Tissue Interaction VIII; 1997: International Society for Optics and Photonics; 1997. p. 115-24.
8. Hemmati F, Kiyani Rad N A. The value of bilicheck(R) as a screening tool for neonatal jaundice in the South of iran. Iranian journal of medical sciences 2013; 38 (2): 122-8.
9. De Luca D, Zecca E, Corsello M, Tiberi E, Semeraro C, Romagnoli C. Attempt to improve transcutaneous bilirubinometry: a double-blind study of Medick BiliMed versus Respironics BiliCheck. Archives of disease in childhood Fetal and neonatal edition 2008; 93 (2): F135-9.
10. Janjindamai W, Tansantiwong T. Accuracy of transcutaneous bilirubinometer estimates using BiliCheck in Thai neonates. Journal of the Medical Association of Thailand=Chotmaihet thangphaet 2005; 88 (2): 187-90.
11. Robertson A, Kazmierczak S, Vos P. Improved transcutaneous bilirubinometry: comparison of SpectR(X) BiliCheck and Minolta Jaundice Meter JM-102 for estimating total serum bilirubin in a normal newborn population. Journal of perinatology: official journal of the California Perinatal Association 2002; 22 (1): 12-4.
12. Polley N, Saha S, Singh S, Adhikari A, Das S, Choudhury B R, Pal S K. Development and optimization of a noncontact optical device for online monitoring of jaundice in human subjects. Journal of biomedical optics. 2015 June; 20 (6): 067001.
13. Alla S K, Huddle A, Butler J D, Bowman P S, Clark J F, Beyette F R. Point-of-care device for quantification of bilirubin in skin tissue. IEEE transactions on biomedical engineering. 2010 Nov. 18; 58 (3): 777-80.

### Example 2

### Multi-Indicator Measurement Wearable Devices

In some embodiments, the wearable device can measure not only bilirubin as described above, but can also measure other health indicators such as oxygen saturation, pulse, breathing rate, serum glucose levels, and skin temperature. Advantageously, no existing devices can measure both bilirubin and such additional health indicators in a single, wearable device. The device as described herein is intended for use on neonates/infants but could be sized appropriately for older subjects.

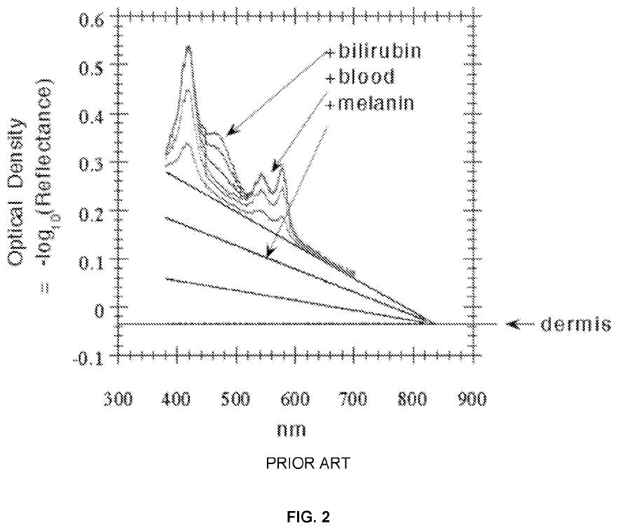

The device can detect multiple biometric signals using a wide-band optical sensor, essentially miniaturizing the reflectance colorimetry methods used in the medical community to detect such signals. Reflectance measurements are influenced by biometric signals across a wide spectrum of visible to near-infrared (NIR) light. A wide-spectrum sensor can differentiate the effects of skin tone (a linear bias), melanin (500-600 nm), and bilirubin (470-490 nm). These effects are shown in FIG. 2. Variations through time allow measurement of pulse and breathing rate (PPG).

Previous research has described the difficulty of obtaining accurate bilirubin and other measurements from a skin-contact device [2, 8]. Bilirubin was measured by measuring the difference between blue (λ=460 nm) and green (λ=570 nm) lights generated with small LEDs. While the project was mildly successful using red and near-infrared light to detect Sp02 and heart rate, bilirubin and other measurements were not accurate enough compared to medical devices.

The present device can provide more accurate measurements by including a spectrometer-integrated circuit with a wide sensing range that is capable of rapid measurement (order of 10 Hz) and a lens/aperture for the wristband optimized to capture data from the skin. In some embodiments, ray-tracing simulation software may be included with the system to improve accuracy. The ray-tracing simulation software can be included in the portable external unit or in the microcontroller of the wearable device.

In some embodiments, the wearable device as described above can be operated without the need for an external portable computational unit. In this embodiment, computation of the data retrieved from the spectrometer is performed in the wearable device itself (e.g., in the microcontroller). The wearable device can be Bluetooth low energy (BLE)-enabled and include a BLE-to-WIFI gateway. A circuit board and microcontroller unit can be included such that the circuit board and microcontroller firmware are configured to collect and retransmit the full spectrometer data set, as well as computing metrics all within the device. The microcontroller then uses BLE to transmit the data and metrics to a BLE central device (e.g., the gateway acts as a central hub and transmits BLE signals from the device to the internet). An associated program, such as a smartphone app, can provide user setup options and provide visual indicators for the metrics and data collected from the device to be interpreted by the user (e.g., a parent or caregiver). The device can be part of a system that includes the wearable device with BLE and a BLE-to-WIFI gateway, a smartphone application, and a server for managing product and customer data. In some embodiments, the device is the wearable only, equipped with BLE connection for sending data directly to a parent/caregiver's smartphone or other device.

In some embodiments, the wearable device is a wristband. The wristband can be comprised of two pieces that connect to each other both mechanically and electrically (such as an upper piece and a lower piece), although other configurations can be envisioned by one of ordinary skill in the art. The first piece can include components such as a circuit board with microcontroller unit, spectrometer integrated circuit and associated LEDs and indicator LEDs. As indicated above, the device is BLE-enabled. The circuit board and microcontroller firmware can be configured to collect and retransmit the full spectrometer data set and may be configured to compute metrics for bilirubin, oxygen saturation, breathing rate, and skin temperature in place from this data. The microcontroller then uses BLE to transmit the data and metrics to a BLE central device. The second piece can be a simple circuit with a battery such as a LiPo rechargeable battery and a charging port.

In some embodiments, a single sensor can be used to detect each of the health indicators (e.g., bilirubin, oxygen saturation, breathing rate, and skin temperature) or multiple sensors can be used to detect health indicators having similar or varying reflectance spectra. Dedicated sensors for oxygen saturation and heart rate can improve battery life and can be used to correlate spectrometer data, and/or correct for environmental factors.

The sensors can provide measurements of a similar quality to existing proven lab spectrometers. The sensors can include such as: AS72651-BLGT, AS7262, AS7265x, or other combination chipsets to form a useful miniaturized spectrometer with an appropriate accuracy and range. These sensors can provide about a 410 to 940 nm range or about a 410 to 2000 nm range with about 20 nm FWHM resolution. Other sensors having similar or better resolution or accuracy may be used, as can be envisioned by one of ordinary skill in the art.

As described above in relation to the bilirubin-only device, a display can also be included instead of or in addition to the data being transmitted to a smartphone application.

The battery will be chosen to balance size and battery life and in some embodiments is targeted to last approximately 24 hours per charge.

In some embodiments, the band pieces of band can be made of skin-safe silicone such that the components are enclosed.

In some embodiments, a simple smartphone application will allow users to view live data from the wristband via BLE. In other embodiments, data from the spectrometer can be stored in a server/gateway and accessed via the smartphone application. Stored data can be used for historical analytics or machine learning.

### REFERENCES

[1] M. Gharmari, “A review on wearable photoplethysmography sensors and their potential future applications in health care,” Int. J. Biosensors & Bioelectronics, vol. 4, no. 4, pp. 195-202, 2018.
[2] G. Inamori, “Neonatal wearable device for colorimetry-based real-time detection of jaundice with simultaneous sensing of vitals,” Applied Sciences and Engineering, vol. 7, no. 10, pp. 1-18, 2021.
[3] D. Bard, “Cuff-less Methods for Blood Pressure Telemonitoring,” Frontiers in Cardiovascular Medicine, vol. 6, 2019.
[4] E. Chan, “Pulse Oximetry: Understanding Its Basic Principles Facilitates Appreciation of Its Limitations,” Respiratory Medicine, vol. 107, no. 6, pp. 789-799, 2013.
[5] O. Cohen, “Glucose Correlation with Light Scattering Patterns—a Novel Method for Noninvasive Glucose Measurements,” Diabetes Technology & Therapeutics, vol. 5, no. 1, pp. 11-17, 2003.
[6] K. Dong Ho and Samsung Electronics Co, “BIO-INFORMATION MEASURING APPARATUS AND BIOINFORMATION MEASURING METHOD”. U.S. Pat. No. 10,772,505, 15 Sep. 2020.
[7] T. Altebaeumer, H. Knoedgen and Bialog Semiconductor, “WEARABLE BIOMETRIC DEVICE AND METHOD OF PERFORMING BIOMETRIC MEASUREMENTS”. U.S. Pat. No. 10,172,557, 8 Jan. 2019.
[8] O. Hiroki, “Bilirubin Concentration Measurement System”. JP Patent 2020/129989, 25 Jun. 2020.
[9] W. Engle, “Transcutaneous Bilirubinometry,” Seminars in Perinatology, vol. 38, pp. 438-451, 2014.
[10] S. Jacques, “Developing an optical fiber reflectance spectrometer to monitor bilirubinemia in neonates,” Laser-Tissue Interactions, vol. 2975, pp. 115-124, 1997.

### Aspects of the Disclosure

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A wearable device for measuring bilirubin levels in a subject, comprising an externally-facing side comprising a display, a skin-facing side comprising at least one light emitting diode (LED) configured to direct light at the subject's skin where the LED is operably connected to a power source, at least one photodetector configured to receive light reflected from the subject's skin, and a processing chip configured to perform spectral subtraction of the reflected light.

Aspect 2. The wearable device of aspect 1, wherein the power source is a battery.

Aspect 3. The wearable device of aspect 2, further comprising a charging port.

Aspect 4. The wearable device of any of aspects 1-3, wherein the device is configured to transmit data wirelessly to an external device.

Aspect 5. The wearable device of aspect 1, wherein the LED light emits light between about 450 nm to 600 nm.

Aspect 6. The wearable device of aspect 1, wherein the device comprises a first LED emitting light between about 415 nm and 488 nm and a second LED emitting light between about 515 nm and 520 nm.

Aspect 7. The wearable device of aspect 1, wherein the device is selected from a bracelet, an anklet, a strap, or a stretchable band.

Aspect 8. The wearable device of aspect 7, wherein the strap or band is dimensioned for wear around one of a user's thigh, arm, waist, or ankle.

Aspect 9. The wearable device of any of aspects 1-5, wherein the device is selected from a wristband, mittens, hat, or socks.

Aspect 10. A wearable device for measuring health indicators in a subject, comprising a skin-facing side, a circuit board, the circuit board comprising a spectrometer integrated circuit, at least one light emitting diode (LED) configured to direct light at the subject's skin where the LED is operably connected to a power source, at least one photodetector configured to receive light reflected from the subject's skin, and a microcontroller configured to perform spectral subtraction of said reflected light; and wherein the health indicators are selected from one or more of: bilirubin levels, pulse, oxygen saturation, breathing rate, and skin temperature.

Aspect 11. The wearable device of aspect 10, wherein the power source is a rechargeable battery.

Aspect 12. The wearable device of aspects 10 or 11, further comprising a charging port.

Aspect 13. The wearable device of aspects 10-12, wherein the device is configured to transmit data wirelessly to an external computing device via bluetooth low energy.

Aspect 14. The wearable device of aspects 10-13, wherein the LED light emits light between about 410 nm to 940 nm.

Aspect 15. The wearable device of aspect 14, wherein the device comprises a first LED emitting light between about 415 nm and 488 nm and a second LED emitting light between about 515 nm and 520 nm.

Aspect 16. The wearable device of aspects 10-15, wherein the device is selected from a bracelet, an anklet, a strap, or a stretchable band.

Aspect 17. The wearable device of aspect 16, wherein the strap or band is dimensioned for wear around one of a user's thigh, arm, waist, or ankle.

Aspect 18. The wearable device of aspect 10, wherein the photodetector is a wide-spectrum sensor and wherein the wide-spectrum sensor differentiates spectral values associated with skin tone, melanin, and bilirubin.

Aspect 19. The wearable device of aspect 18, wherein the wide-spectrum sensor detects a range of about 410 nm to 2000 nm with a resolution of about 20 nm FWHM.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A wearable device for measuring bilirubin levels in a subject, comprising:
an externally-facing side comprising a display;
a skin-facing side comprising at least one light emitting diode (LED) configured to direct light at the subject's skin where the LED is operably connected to a power source;
at least one photodetector configured to receive light reflected from the subject's skin; and
a processing chip configured to perform spectral subtraction of the reflected light.

2. The wearable device of claim 1, wherein the power source is a battery.

3. The wearable device of claim 2, further comprising a charging port.

4. The wearable device of claim 1, wherein the device is configured to transmit data wirelessly to an external device.

5. The wearable device of claim 1, wherein the LED light emits light between about 450 nm to 600 nm.

6. The wearable device of claim 1, wherein the device comprises a first LED emitting light between about 415 nm and 488 nm and a second LED emitting light between about 515 nm and 520 nm.

7. The wearable device of claim 1, wherein the photodetector is a wide-spectrum sensor and wherein the wide-spectrum sensor differentiates spectral values associated with skin tone, melanin, and bilirubin

8. The wearable device of claim 1, wherein the device is selected from a bracelet, an anklet, a strap, or a stretchable band.

9. The wearable device of claim 8, wherein the strap or band is dimensioned for wear around one of a user's thigh, arm, waist, or ankle.

10. The wearable device of claim 1, wherein the device is selected from a wristband, mittens, hat, or socks.

11. A wearable device for measuring health indicators in a subject, comprising:
a skin-facing side comprising a circuit board, the circuit board comprising
a spectrometer integrated circuit, at least one light emitting diode (LED) configured to direct light at the subject's skin where the LED is operably connected to a power source, at least one photodetector configured to receive light reflected from the subject's skin, and a microcontroller configured to perform spectral subtraction of said reflected light; and
wherein the health indicators are selected from one or more of: bilirubin levels, pulse, oxygen saturation, breathing rate, and skin temperature.

12. The wearable device of claim 11, wherein the power source is a rechargeable battery.

13. The wearable device of claim 11, further comprising a charging port.

14. The wearable device of claim 11, wherein the device is configured to transmit data wirelessly to an external computing device via bluetooth low energy.

15. The wearable device of claim 11, wherein the LED light emits light between about 410 nm to 940 nm.

16. The wearable device of claim 15, wherein the device comprises a first LED emitting light between about 415 nm and 488 nm and a second LED emitting light between about 515 nm and 520 nm.

17. The wearable device of claim 11, wherein the device is selected from a bracelet, an anklet, a strap, or a stretchable band.

18. The wearable device of claim 17, wherein the strap or band is dimensioned for wear around one of a user's thigh, arm, waist, or ankle.

19. The wearable device of claim 11, wherein the photodetector is a wide-spectrum sensor and wherein the wide-spectrum sensor differentiates spectral values associated with skin tone, melanin, and bilirubin.

20. The wearable device of claim 19, wherein the wide-spectrum sensor detects a range of about 410 nm to 2000 nm with a resolution of about 20 nm FWHM.
